(19) United States
(12) Patent Application Publication
(10) Pub. No.: US 2006/0263837 A1
(43) Pub. Date: Nov. 23, 2006

(54) Immunoassay system and method for detection of antigens

(76) Inventor: George Dacai Liu, Collegeville, PA (US)

Correspondence Address: DR. GEORGE DACAI LIU, 1029 BRASSINGTON DRIVE, COLLEGEVILLE, PA, 19426, US

(21) Appl. No.: 11/493,482

(22) Filed: Jul. 26, 2006

Related U.S. Application Data
(63) Continuation-in-part of application No. 10/870,766, filed on Jun. 17, 2004.

(51) Int. Cl. G01N 33/543

(52) U.S. Cl. 435/7.93; 436/518

(57) ABSTRACT

The present invention provides a system and method for detecting antigens without discrimination between antigens. The present invention further provides a diagnostic kit for detecting markers derived from a disease or clinical samples.

### FIELD OF THE INVENTION

The present invention generally relates to immunoassays, and more particularly to immunoassay systems and methods that detect antibody-bound antigens without discrimination between antigens.

### BACKGROUND OF THE INVENTION

Immunoassays, including enzyme-linked immunosorbent assay (ELISA) and western blot, have been broadly used in research and diagnostics. With respect to a classic ELISA, one first single monoclonal antibody (mAb) is coated upon one single well in an assay plate, and then contacted with a sample that may contain the antigen recognizable by the coated antibody; then one second antibody is added for detection of the bound antigen where the second antibody recognizes the same antigen but at a different epitope. Thus, a pair of mAbs or one mAb and polyclonal antibodies is needed for each antigen. This makes it impossible to use this ELISA format when only one mAb for one antigen is available.

One modification to overcome the requirement of a pair of antibodies for each antigen is to chemically label the antigens directly by some detectable molecules such as fluorescence, radioisotopes, and tags (e.g., biotin). However, the labeling process itself is cumbersome and time-consuming. Furthermore, diversified proteins cannot be labeled uniformly or consistently, resulting in inter-/intra-experiment variations. In addition, the labeling molecules may interfere with the formation of antigen-antibody complex.

Both classical ELISA and chemically labeling schemes have been employed in antibody arrays for assaying multiple proteins simultaneously as tools for proteomics, drug discovery and pharmacoproteomics. The antibody arrays are comprised of a plurality of mAbs where the mabs are usually spotted into rows/columns. Currently, the captured proteins in an antibody array are detected by the same schemes used for single antibodies. One is a sandwich immunoassay in which capture antibodies are immobilized on the solid support, and the bound proteins are detected using a mixture of second, labeled detection antibodies. For example, Huang et al. (Anal. Biochem. 2001, 294:55-62) described an ECL based immunoassay array for the simultaneous assay of 24 cytokines from either cultured media or patient sera. The system was based on the standard sandwich ELISA technology but the initial capture antibodies raised to the various cytokines were transferred in an ordered format onto a membrane. It is evident that this type of antibody array is a simple collection of multiple individual ELISA assays. Another is an antigen capture assay in which proteins are similarly captured by immobilized antibodies, but the captured proteins are detected directly. For example, Haab et al. (Genome Biol., 2001, 2research0004.1-0004.13) labeled two samples independently with distinguishable fluorophores and mixed the samples before applying them to the array. However, this method does not offer signal amplification beyond that provided by the use of fluorophores. In addition, the labeling is time consuming and some proteins may be labeled preferentially on their antigenic epitopes and lose their ability to be captured by their affinity reagents.

### SUMMARY OF THE INVENTION

The present invention provides a system and method for detecting antigens without discrimination between antigens. In one embodiment, the present invention provides a method for detecting antigens captured on an antibody. The method comprises the following steps of providing at least one IgG monoclonal antibody; contacting the at least one IgG monoclonal antibody with a sample containing at least one antigen, wherein the at least IgG monoclonal antibody is specific for the at least one antigen; and detecting the at least one antigen captured by the at least one IgG monoclonal antibody with a detecting agent that differentially binds to the at least one IgG monoclonal antibody when it binds to the at least one antigen over the at least one IgG monoclonal antibody when it is empty; thereby when the at least one antigen binds to the at least one IgG monoclonal antibody, the detecting agent binds to the at least one IgG monoclonal antibody differentially; thus the at least one antigen can be detected by the detecting agent without discrimination between antigens.

In another embodiment of the method, the at least one IgG monoclonal antibody is from species selected from the group consisting of mouse, rat, rabbit, sheep, goat, horse, bovine, dog and human. In another embodiment of the method, the at least antigen is one selected from the group consisting of proteins, peptides, glycoproteins, lipoproteins, lipids, nucleic acids, small molecules, allergens, viruses, bacteria, yeasts, fungi, unicellular/multicellular organisms, and cells.

In another embodiment of the method, the sample includes a clinical sample, and cultured cells, cell supernatants, cell lysates, serum, plasma, biological fluid, and a pure or enriched bacterial or viral sample derived from any of these. In another embodiment of the method, the detecting agent is a protein or antibody or, portions or fragments thereof. In another embodiment of the method, the detecting agent is Clq or, portions or fragments thereof. In another embodiment of the method, the at least one IgG monoclonal antibody forms an array with two or more different IgG monoclonal antibodies.

Another embodiment of the present invention provides a system for detecting antigens captured on at least one IgG monoclonal antibody. The system comprises at least one IgG monoclonal antibody, wherein the at least one IgG monoclonal antibody is specific for at least one antigen; and a detecting agent that differentially binds to the at least one IgG monoclonal antibody when it binds to the at least one antigen over the at least one IgG monoclonal antibody when it is empty, thereby when the at least one antigen binds to the at least one IgG monoclonal antibody, the detecting agent binds to the at least one IgG monoclonal antibody differentially; thus the at least one antigen can be detected by the detecting agent without discrimination between antigens.

In another embodiment of the system, the at least one IgG monoclonal antibody is from species selected from the group consisting of mouse, rat, rabbit, sheep, goat, horse, bovine, dog and human. In another embodiment of the system, the at least one antigen is one selected from the group consisting of proteins, peptides, glycoproteins, lipoproteins, lipids, nucleic acids, small molecules, allergens, viruses, bacteria, yeasts, fungi, unicellular/multicellular organisms, and cells. In another embodiment of the system, the detecting agent is a protein or antibody or, portions or fragments thereof. In another embodiment of the system, the detecting agent is Clq or, portions or fragments thereof.

In another embodiment of the system, the at least one IgG monoclonal antibody forms an array with two or more different IgG monoclonal antibodies.

Another embodiment of the present invention provides a diagnostic kit for detecting markers derived from a disease. The diagnostic kit comprises at least one IgG monoclonal antibody, wherein the at least one IgG monoclonal antibody is specific for at least one disease marker; and a detecting agent that differentially binds to the at least one IgG monoclonal antibody when it binds to the at least one disease marker over the at least one IgG monoclonal antibody when it is empty; thereby when the at least one disease marker binds to the at least one IgG monoclonal antibody, the detecting agent binds to the at least one IgG monoclonal antibody differentially, thus the at least one disease marker can be detected by the detecting agent without discrimination between disease markers. In another embodiment, the diagnostic kit further comprises an instruction sheet.

In another embodiment of the diagnostic kit, the detecting agent is Clq or an antibody, portions or fragments thereof. In another embodiment of the diagnostic kit, the at least one IgG monoclonal antibody forms an array with two or more different IgG monoclonal antibodies.

The objectives and advantages of the invention will become apparent from the accompanying drawings and the following detailed description of preferred embodiments thereof.

### BRIEF DESCRIPTION OF THE DRAWINGS

FIG. 1 shows the graphs of classical ELISA results of ten monoclonal antibodies.

FIG. 2 shows the graphs of Clq-ELISA results of ten monoclonal antibodies.

FIG. 3 shows the results of Clq-ELISA on slides.

### DETAILED DESCRIPTION OF THE INVENTION

The present invention may be understood more readily by reference to the following detailed description of certain embodiments of the invention.

Throughout this application, where publications are referenced, the disclosures of these publications are hereby incorporated by reference, in their entireties, into this application in order to more fully describe the state of art to which this invention pertains.

The present invention provides systems and methods of detecting antibody-bound antigens without discrimination between antigens, where the “antibody-bound antigen” refers to an antigen that has been captured by a solid-supported antibody. The practice of the present invention will employ, unless otherwise indicated, conventional techniques of molecular biology (including recombinant techniques), microbiology, cell biology, biochemistry and immunology, which are known to those skilled in the art. For example, monoclonal and polyclonal antibodies can be produced for different antigens in different hosts by known methods. The manipulation of the antibodies is also known. In addition, the production and preparation of antigens are also known to those skilled in the art. Thus, no citation to or detailed description of the known techniques will be given herein for the sake of brevity.

It is common that one molecule or one class of molecules can be induced to perform certain similar, if not identical, function(s) by the interactions (e.g., binding) with different entities, regardless of the structures of the entities interacted with the molecule(s). The typical molecules are the antibodies in animals including mouse, rat, rabbit, pig, goat, horse, dog and human beings. For example, when IgGs are bound by their antigens, the IgGs will go conformational changes so that the first component of the classical complement pathway, Clq, can bind to the IgGs, resulting in the activation of the classical complement pathway. It is reasoned that all the antigen-bound IgGs must share at least one conformational epitope that is recognized by the Clq. More importantly, different antigens can induce the shared conformational epitopes regardless of the antigen structures. Therefore, an antigen bound to its corresponding antibody can be detected by detecting of the conformational changes on the antibody instead of the identity of the antigen. One of the evident applications of this finding is to antibody arrays wherein antigens captured by the immobilized antibodies can be detected by a single or few detecting agents that recognize the shared conformational epitopes on the antibodies. The obvious advantages include no more requirements of paired antibodies for each antigen and no more cocktails of second antibodies for each antigen to be detected on an antibody array.

Monoclonal Ab array is usually comprised of monoclonal immunogloblins G (IgGs). It is well known that an IgG molecule is bi-functional with two distinct regions: variable (V) region and constant (C) region. Murine IgG includes four subclasses, IgG1, IgG²a, IgG2b and IgG3. While each IgG has a different V region for recognizing a different antigen, each subclass has an identical C region for mediating effector functions including binding of IgG to host tissues, to various cells of the immune system, and to the first component (Clq) of the classical complement system. In view of this, we reasoned that IgG molecules as a whole could be viewed as intra-molecular signal translators—translating the different signals from the V regions into identical or similar signals to the C regions for effector functions. Then we hypothesized that IgGs themselves held the key to the detection of different antigens captured on a mAb array without discrimination between antigens if we could find a molecule that could detect the unified signals induced by different antigens.

Throughout the present application, antibody, antigen, antibody array and detecting agents are used for the simplicity of description. “Antibody” as used herein is intended to cover any molecule or any class of molecules on which at least one shared conformational epitope can be induced by two or more different antigens. It is to be noted that the definition is function-based. While the examples illustrate the induction of new conformational epitopes recognized by Clq, it is to be appreciated that the loss of conformational epitopes on antigen-bound antibodies may also be utilized to detect and/or verify the binding of antigens. The induced or lost conformational epitopes on an antigen-bound antibody shared by at least two antibodies are referred herein as “shared conformational epitopes.” An applicable antibody may include conventional antibodies, antibody minics and receptors. The conventional antibodies can encompass monoclonal, polyclonal antibodies, chimeric antibodies, single chain, and mutants thereof. Antibodies may be murine, rat, rabbit, chicken, human, or any other origin (including humanized antibodies). General techniques for antibodies are known in the art.

“Antigen” as used herein refers to any entity that binds to an antibody disposed on an antibody array and induces at least one shared conformational epitope on the antibody. Antigens could be proteins, peptides, antibodies, small molecules, lipid, carbohydrates, nucleic acid, allergens, viruses, bacteria, fungi, and/or the like. An antigen may be in its pure form or in a sample in which the antigen is mixed with other components.

“Antibody array” as used herein refers to a linear or two-dimensional array of two or more different antibodies formed on the surface of a solid support.

“Detecting agent” as used herein refers to any molecule that has the ability to bind to at least one shared conformational epitope (resulting from the binding of antigens) of immobilized antibodies. The detecting agent will not bind, or bind at an insignificant level the antibodies immobilized on an antibody array if the antibodies are not bound with their correspondingly antigens. Or the difference of the detecting agent's bindings between antigen-bound antibody and empty antibody is statistically differential. The shared conformational epitopes may be new ones induced by the antigens or lost ones destroyed by the antigens. The detecting agent includes proteins (e.g., Clq, RF), peptides (Clq fragments), and antibodies that specifically recognize the shared conformational epitopes. The detecting agent may preferentially bind to empty antibody over loaded ones. In other cases, the detecting agent may preferentially bind to loaded antibodies over empty ones.

In one embodiment of the present invention, there is provided an antibody system for detecting antigens captured on the antibodies. The system comprises one or more antibodies for antigens, and at least one detecting agent that is able to recognize at least one shared conformational epitope on the antibodies resulting from the binding of the antigens. When one antibody is used, the antibody can be disposed onto any solid support including wells, slides, spheres, and membranes. When two or more antibodies are used, the antibodies can be disposed onto any solid support in such a way that their signals can be detected separately. In one embodiment, the two or more antibodies form an antibody array.

An antibody array is an ordered spatial arrangement of two or more antibodies on a physical substrate. Row and column arrangements are preferred due to the relative simplicity in making and assessing such arrangements. The spatial arrangement can, however, be essentially any form selected by the user, and preferably but need not be, in a pattern. The most common form of antibody arrays is that antibodies that bind specific antigens are arrayed on a glass slide at high density. A sample containing possible antigens is passed over the array and the bound antigen is detected after washing.

Antibodies are preferably printed onto a solid support. Amongst the large number of solid-support materials applicable for the production of antibody arrays, silica or glass is most often used because of its great chemical resistance against solvents, its mechanical stability, its low intrinsic fluorescence properties, and its flexibility of being readily functionalized. Examples of well known solid supports include polypropylene, polystyerene, polyethylene, dextran, nylon, amylases, glass, natural and modified celluloses, polyacrylamides, agaroses and magnetite. Those skilled in the art will know of other suitable solid support for binding antibodies, or will be able to ascertain such, using routine experimentation.

Antibodies may be immobilized onto a support surface either by chemical ligation through a covalent bond or non-covalent binding. There are many known methods for covalently immobilizing antibodies onto a solid support. For example, MacBeath et al. (J Am Chem Soc, 1999, 121:7967-7968) use Michael addition to link thiol-containing compounds to maleimide-derivatized glass slides to form a microarray of small molecules. See also, Lam and Renil, Current Opinion in Chemical Biology, 2002, 6:353-358.

Antibodies may be attached to various kinds of surface via diffusion, adsorption/absorption, covalent cross-linking and affinity. Antibodies may be directly spotted onto plain glass surface. To keep antibodies in a wet environment during the printing process, high percent glycerol (30-40%) may be used in sample buffer and the spotting is carried out in a humidity-controlled environment.

The surface of a substrate may be modified to achieve better binding capacity. For example, the glass surface may be coated with a thin nitrocellulose membrane or poly-L-lysine such that antibodies can be passively adsorbed to the modified surface through non-specific interactions. In addition, streptavidin may be arrayed onto solid surfaces for capture of biotinylated proteins.

Antibody arrays can be fabricated by the transfer of antibodies onto the solid surface in an organized high-density format followed by chemical immobilization. The techniques for fabrication of an array include, but are not limited to, photolithography, ink jet and contact printing, liquid dispensing and piezoelectrics. The patterns and dimensions of antibody arrays are to be determined by each specific application. The sizes of each antibody spots may be easily controlled by the users.

As discussed above, the present invention takes advantage of the shared conformational epitopes on antibodies resulting from the binding of antigens. The shared conformational epitopes can be induced or destroyed by different antigenic epitopes from one antigen or different antigenic epitopes from different antigens. The detecting agent of the present invention is capable of recognizing at least one shared conformational epitope present in all antigen-bound antibodies so that the detection of antigens bound to antibodies is without discrimination between antigens, where the antigen-bound antibodies refer to the antibodies that have captured their antigens. The nature of the detecting agent is not important for the present invention as long as the detecting agent is applicable for the present invention.

In one embodiment, the present invention provides the detecting agents including complement lq (Clq) and rheumatoid factor (RF). Both Clq and RF have the property of binding to antibodies that are part of complexes formed between antibodies and antigens, but essentially do not bind to non-complexed antibodies. Both Clq and RF have been used to measure circulating immune complexes. See, U.S. Pat. No. 4,143,124; and PCT, WO 97/01758. Clq is the first component of the classical complement cascade pathway, which is commonly present in animals including mouse, rat, rabbit, sheep, goat, horse, cattle, dog and human beings. Clq shares high homology with similar structures from different species and has cross species activities. Thus, Clq applicable in the present invention is not limited to any Clq from a specific species. For example, human Clq is a glycoprotein of about 460 kDa. In its electron microscopy image Clq appears as a bunch of tulips, with six globular heads, each connected by a stalk to a central bundle of fibers. One Clq molecule is composed of 18 polypeptide chains. The chains are of three different types named A, B, and C, of 29, 27, and 23 kDa, respectively. They are linked by disulfide bonds to form six A-B and three C-C dimers. Each of the six individual segments of Clq comprises one chain of each type, which acquires a triple helical structure in the fibrillar region. See, e.g., Kaul and Loos, The Journal of Biological Chemistry, 1997, 272:33234-33244.

Clq binds to antibodies in antigen-antibody complexes. As for IgG, Clq may bind to empty antibodies when antibodies are coated onto wells in high concentrations or in the form of heat-denatured aggregates. The binding of Clq to antibodies may be optimized by exploring concentration, incubation time, incubation temperature, pH values, and ionic strengths. The optimized conditions for each antibody array can be obtained with methods well known to those skilled in the art. See, Tan et al., Proc. Natl. Acad. Sci. USA, 1990, 87:162-166; Marques et al., The Journal of Biological Chemistry, 1993, 268:10393-10402.

In addition, some portions or fragments of Clq have retained the capacity of binding to antigen-bound antibodies. Thus, Clq as used herein includes any Clq portions or fragments that retain the capacity of binding to antigen-bound antibodies. Fragments of Clq and peptides prepared synthetically are described in WO 92/07267.

Rheumatoid factors (RF) are antiglobulin antibodies that bind IgG immunoglobulins and are found in the serum and synovial fluid of most patients with rheumatoid arthritis. Certain RFs can bind to neoantigens created within IgG by the formation of antigen-antibody immune complexes. See, U.S. Pat. No. 5,252,461. RF is a known material and methods for its preparation and isolation is known. See, U.S. Pat. No. 4,143,124. A portion or fragment of RF can also be used for this invention if it retains its binding capacity. See, PCT/DK96/00288.

In another embodiment of the present invention, there is provided methods for detecting the antigens bound to the antibodies including the antibody arrays as described hereinabove. Briefly, the methods comprise the steps of providing one or more antibodies, contacting the antibodies with a sample containing antigens, and detecting the bound antigens by using the detecting agents capable of specifically, selectively or differentially binding to antigen-bound antibodies. The process can be done manually and/or automatically. The handling of arrays is well known to those skilled in the art.

As used herein, “sample” encompasses a variety of sample types and/or origins, such as blood and other liquid samples of biological origin, solid tissue samples such as a biopsy specimen or tissue cultures or cells derived therefrom, and the progeny thereof. The term “sample” encompasses a clinical sample, and also includes cells in culture, cell supernatants, cell lysates, serum, plasma, biological fluid, and a pure or enriched bacterial or viral sample derived from any of these, for example, as when a sample is cultured in order to increase, enrich and/or substantially purify a bacterial or viral sample therefrom. A sample can be from microorganisms, e.g., bacteria, yeasts, viruses, viroids, molds, fungi, plants, animals, including mammals such as humans. A sample may comprise a single cell or more than a single cell. These samples can be prepared by methods known in the art such as lysing, fractionation, purification, including affinity purification, FACS, laser capture microdissection or iospycnic centrigugation.

When the antibody is contacted with a sample, the formation of antibody:antigen complexes can be performed under a variety of conditions. It is also true for the reaction of the detecting molecules with the antibodies. The reaction solutions can contain varying degrees of salt or have varying pH values. In addition, the binding reaction can be carried out at varying temperature. In general, pH conditions will range from 2-10 (most preferably around pH7), temperatures from 4-45° C. and salt conditions from 1 μm to 5M (in the case of NaCl).

The readout of the detecting agents bound to the antibodies in an antibody array can take up many forms. Prior to description of the readout methods, it is to be appreciated that the antigens in a sample and the secondary detecting molecules (e.g., antibodies against the detecting molecules such as anti-Clq antibodies) against the detecting agents can be detectably labeled and detected as the detecting agents as described hereinafter. For example, the antigens in a sample can be labeled with fluorescent dyes or detected by their specific antibodies in combination with the detecting agents to quantify and/or verify the binding antigens. Therefore, the detection methods for detecting agents may be compatible with other immunoassays as users desire.

The term “detectably labeled” as used herein is intended to encompass detecting agent directly coupled to a detectable substance, such as a fluorescent dye, and detecting agent coupled to a member of binding pair, such as biotin/streptavidin, or an epitope tag that can specifically interact with a molecule that can be detected, such as by producing a colored substrate or fluorescence.

Fluorescence detection methods are generally the preferred detection method because they are simple, safe, extremely sensitive and can have very high resolution. Typically, an antibody is either directly probed with a fluorescent detecting molecule or in two steps by first using a tagged probe (e.g., biotin), which can then be detected in a second step using a fluorescently labeled affinity reagent (e.g., streptavidin).

A biotin labeled target can be detected by gold-conjugated streptavidin with silver enhance solution so that the resultant black image of microarray spots can be easily detected with a commercial CCD camera.

The detecting agents (e.g., Clq and RF) and anti-detecting agent antibodies may be attached by the 5′ end of an oligonucleotide primer. Then the signals may be significantly increased by Rolling circle amplification (RCA). In the presence of a DNA circle, DNA polymerase and nucleotides, rolling circle replication generates a concatamer of circle DNA sequence copies that remain attached to the antibody. The concatamer is then detected by the hybridization of fluorescent, complementary oligonucleotide probes. See, e.g., Schweitzer et al., Proc Natl Acad Sci USA 2000, 97:10113-10119.

Clq that binds to the antibody array can be detected using any means known in the art. In some embodiments, the Clq is labeled, using any methods known in the art. See, U.S. Pat. No. 4,882,423. For example, the Clq may be labeled with one or more labeling moieties including compositions that can be detected by photochemical, spectroscopic, biochemical, immunochemical, chemical, optical, electrical, bioelectronic, etc. means. For example, useful protein labels include radiolabels (e.g., 3H, 125I, 35S, 14C, or 32P), fluorescent dyes (e.g., fluorescein isothiocyanate, Texas red, rhodamine, and the like); electron-dense reagents, enzymes (e.g., LacZ, CAT, horse radish peroxidase, alkaline phosphatase and others, commonly used as detectable enzymes, either as marker gene products or in an ELISA); biotin, dioxigenin, or haptens and proteins for which antisera or monoclonal antibodies are available. A wide variety of labels and conjugation techniques are known and generally applicable to the present invention for the labeling of proteins.

Analysis of antigens bound to the antibody arrays can be quantitative, semi-quantitative or qualitative. “Detect” refers to identifying the presence, absence and/or amount of antigen to be detected. “Absence” of binding, and “lack of detection of product” as used herein includes insignificant or de minimus levels.

It is to be appreciated that the antibodies in the present invention need not be characterized in terms of their specificities for antigens because no paired antibodies are required for the detection of the bound antigens in accordance with the present invention. In certain embodiments, arrays of uncharacterized antibodies may be used to compare the protein expression profiles of cells, for example, comparisons can be made between a population of cells from one tissue, and a second tissue, or from cells derived from a particular tissue but from different species. Comparisons can be made between normal cells and cells from the same tissue type that originate from an individual with a pathogenic disorder. For example, comparisons can be made between normal cells and cancer cells. Comparisons can additionally be made between cells in a resting state and cells in an activated state.

Detection and characterization of bacterial or viral infection is of crucial importance in the practice of clinical microbiology and in environmental testing, such as food safety and biohazard safety testing. In another embodiment, the disclosed arrays are useful for evaluating the expression of proteins by pathogens, such as, for example, bacteria, parasites, viruses, and the like. These antibodies have utility as diagnostic agents as well as potential therapeutics.

The systems and methods disclosed herein can be used in methods of diagnosing particular disorders. For example, in a diagnostic kit, a collection of antibodies specific for a range of antigens associated with one or more disorders can be arrayed and contacted with a bodily fluid containing antigens whose presence or absence would indicate a particular disorder. The advantage of using an array over a conventional immunoassay is the ability to include a population of antibodies diagnostic for a variety of disorders on a single surface, significantly reducing time, costs and materials needed to effect a diagnosis.

It is to be appreciated that many conventional procedures are not described herein including blocking and washing steps in performing the methods of the present invention. These procedures are well known to those in the art.

### EXAMPLES

The following examples are offered by way of illustration and not by way of limitation.

### Example 1

Murine monoclonal antibodies were raised against recombinant human prion proteins. Their specificity for recombinant prion proteins (PRP) including human and bovine was verified by ELISA and western blot. The antibodies were purified in accordance with standard techniques. A total of seven murine monoclonal antibodies against recombinant human PRP were used in the experiments described herein. Antibodies (7A12, 8B4, 2C2, 8H4 and 6H3) are IgG1 and antibodies (8F9 and 12H7) are IgG2b. Murine monoclonal antibodies against IL-2 and IL-4 were commercially available. Human Clq and Sheep anti-human Clq were purchased from Cortex Biochem Inc. (San Leandro, Calif.). Donkey anti-sheep IgG-HRP was purchased from Roche Applied Science (Indianapolis, Ind.).

The first experiment was intended to demonstrate that the immobilization of antibodies on the solid support does not cause Clq to bind to the immobilized antibodies, and to show that Clq is able to detect the specific binding of antigens to their antibodies.

The experiments described herein were done in accordance with conventional ELISA assays except for that the second antibody was substituted with Clq. Briefly, the antibodies were diluted in PBS (pH 7.4) to a concentration of 2 μg/ml. Each well of a 96-well plate was coated with 100 μl of diluted antibodies (anti-IL2, anti-IL4, and 7A12) for 3 hours at room temperature. Then coated wells were blocked with commercial blocking buffer for 1.5 hours at room temperature. After washing three times with PBST (PBS with 0.05% Tween-20), recombinant human prion proteins were added into each well (100 μl/well) at concentration of 2 μg/ml, and incubated for overnight at 4° C. After washing three times with PBST, human Clq was added into each well at concentrations of 2 μg/ml, 0.2 μg/ml, 0.02 μg/ml and 0 μg/ml and incubated for three hours at room temperature. After washing three times with PBST, sheep anti-human Clq antibodies were added into each well at concentration of 2 μg/ml and incubated for 1.5 hours at room temperature. After washing three times with PBST, donkey anti-sheep antibodies conjugated with HRP were added into each well (1/5000) and incubated for 1.5 hours at room temperature. Finally the wells were developed with ABTS for thirty minutes. The results were summarized in Table 1.

TABLE 1
Summary of OD reading under the conditions described above.

| | ABs | | |
|---|---|---|---|
| [C1q] | Anti-IL2 | Anti-IL4 | 7A12 |
| 0 | 0.085 | 0.1 | 0.115 |
| 0.02 | 0.09 | 0.1 | 0.155 |
| 0.2 | 0.09 | 0.1 | 0.16 |
| 2 | 0.09 | 0.11 | 0.2 |

### Example 2

More anti-human PRP antibodies were tested for their conformational changes induced by their antigens. The procedure was the same as Example 1 except for that recombinant human prion proteins were added into each well (100 μl/well) at concentrations of 2 μg/ml, 0.2 μg/ml and 0 μg/ml, and that the Clq was tested in three concentrations instead of four.

The results are summarized in Table 2. The results demonstrated that Clq is able to distinguish the immobilized but free antibodies from the immobilized but bound with antigens. It is to be noted that some antibodies may not be suitable for the application of antibody arrays. As shown here, 8F9 is not suitable for such purpose. However, whether an antibody is suitable for the application of antibody array may be easily determined by routine experimentation.

TABLE 2
Summary of OD reading under the conditions as described above.

| | Abs | | | | | | | | |
|---|---|---|---|---|---|---|---|---|---|
| | 8B4 | | | 2C2 | | | 8H4 | | |
| | C1q | | | | | | | | |
| PRP | 0 | 0.2 | 2 | 0 | 0.2 | 2 | 0 | 0.2 | 2 |
| 0 | 0.041 | 0.054 | 0.048 | 0.059 | 0.057 | 0.066 | 0.145 | 0.147 | 0.156 |
| 0.2 | 0.041 | 0.092 | 0.218 | 0.067 | 0.185 | 0.506 | 0.141 | 0.22 | 0.294 |
| 2 | 0.043 | 0.08 | 0.22 | 0.066 | 0.152 | 0.441 | 0.14 | 0.169 | 0.222 |
| | Abs | | | | | | | | |
| | 8F9 | | | 6H3 | | | 12H7 | | |
| | C1q | | | | | | | | |
| PRP | 0 | 0.2 | 2 | 0 | 0.2 | 2 | 0 | 0.2 | 2 |
| 0 | 0.055 | 0.251 | 0.731 | 0.115 | 0.125 | 0.124 | 0.131 | 0.138 | 0.144 |
| 0.2 | 0.054 | 0.248 | 0.724 | 0.118 | 0.147 | 0.183 | 0.128 | 0.341 | 0.817 |
| 2 | 0.052 | 0.273 | 0.732 | 0.125 | 0.15 | 0.187 | 0.090 | 0.236 | 0.828 |

### Example 3

Methods

Antigens, antibodies, and detection reagents. Antigens included two subtypes of avian influenza viruses (AIV) (H5N1 and H9N2), and foot-mouth disease virus (FMDV)). H5N1 AIV was isolated from healthy duck in Guangdong province (P R China) in 2000, while H9N2 AIV was isolated from infected chicken in Guangdong province in 2001. FMDV used in this study was type O strain. AIVs were propagated for 28 to 48 hours in the allantoic cavities of 10-day-old embryonated chickens' eggs at 37° C., and FMDV were propagated in BHK21 cells. All viruses were purified by equilibrium density centrifugation through a 30 to 60% linear sucrose gradient as described. The concentrations of viral protein were measured by Bradford's methods.

Murine monoclonal antibodies included four IgG1 mAbs (IE6, IIIC2D2-01, IIB6C3, 2D5H12), two IgG2a mAbs (IIA8H11-04, IIG6A11), two IgG2b mabs (H901, IIIF6G8-03), and two IgM mAbs (IIID6D6-02, D2H11). IIID6D6-02 and 2D5H12 are specific for FMDV; H901 is specific for H9N2; and the rest mAbs are specific for H5N1. All monoclonal antibodies were generated and produced as ascites following conventional protocols for making monoclonal antibodies as described. Some ascites were purified prior to use using Protein A Sepharose™ CL-4B (Amersham Biosciences, USA).

Chicken anti-H5N1 AIV serum and chicken anti-H9N2 AIV serum were provided by Harbin Veterinary Research Institute (PRC), and cavy anti-FMDV serum was provided by Lanzhou Veterinary Research Institute (PRC). HRP-conjugated rabbit anti-chicken IgG and HRP-conjugated rabbit anti-cavy IgG were purchased from Guangzhou Boli Biotech Co Ltd (PRC).

Human Clq protein was from Alpha Diagnostic International (USA). Sheep anti-human Clq polyclonal antibody was from Serotec Ltd (USA). Rabbit anti-sheep IgG polyclonal antibody was from Shenzhen Jingmei Biotech Ltd (PRC).

Clq-Based Detection of antigens in a sandwich ELISA-like format (Clq-ELISA).

Clq-ELISA protocol was very similar to the one of a typical sandwich ELISA (see Supplementary Information) except that Human Clq substituted the second antibody. Briefly, mAbs were diluted in coating buffer and distributed in each well (100 μl) of a 96-well plate. The plate was incubated overnight at 4° C., washed 3× using PBST, blocked with blocking buffer at 4° C. for 5 hours, and washed again with PBST. Antigens were diluted in dilution buffer, and added into each well. The plate was incubated at room temperature for 2 hours, and similarly washed as above. The plate was added with Clq and incubated overnight at 4° C. Then, the plate was washed with washing buffer, and sheep anti-human Clq polyclonal antibodies were added. After incubation at room temperature for 1 hour, the plate was washed and incubated with HRP-conjugated rabbit anti-sheep IgG polyclonal antibodies at room temperature for 1 hour. Finally, the plate was washed, and incubated with TMB substrate. The reaction lasted for 5-10 minutes, stopped, and the OD was read at 450 nm.

Statistical analysis. All analyses were performed using the statistical package SAS 9.0 (SAS Institute, Cary, N.C., USA). Two-way ANOVA was used to assess OD differences of different antigen concentration levels over the control (Empty—no antigen). The statistical difference was tested under four conditions (a=0.05, 0.01, 0.005, and 0.001).

Clq-based detection of antigens on mAb arrays spotted on glass slides (Clq-mAb array).

The glass slides were provided by Shenzhen Sciarray Biochip Ltd (PRC), where the glass slides were first treated with the amido silane and then coated with aldehyde-activated agarose. Each slide had a spotting area of 1×1 cm. Briefly, IE6, IIIC2D2-01, and 2D5H12 were diluted in the CBS with 30% glycerol into 40 μg/ml, and 1 μg of the solution was spotted using Biochip Arrayer (Axon, USA); and the spotted slides were incubated within a humidified box overnight at 4° C. After three times of washing with PBST (0.05% Tween 20), the slides were blocked with blocking buffer (3% BSA) at room temperature for 2 hours. After 3 times of washing, antigens were added to the slide and incubated at room temperature for 2 hours. After 3 times of washing, 200 μl of human Clq (2 μg/ml) was added to the slide and incubated overnight at 4° C. After washing off the unbound Clq, sheep-anti-human Clq polyclonal antibodies (1:200 dilution) was added and incubated at room temperature for 1 hour. After washing, HRP-conjugated rabbit anti-sheep IgG polyclonal antibodies (1:2000 dilution) was added and incubated at room temperature for 1 hour. After washing off unbound rabbit anti-sheep IgG antibodies, substrate solution was added, and the coloring was allowed to develop for 3-5 minutes before the slides were photographed.

Optimizing conditions of Clq-mAb array. The basic protocol for Clq-mAb array is similar to that of Clq-ELISA. Briefly, 1 μl of mAb diluted in the fixing buffer was manually spotted for each spot. Then the spotted glass slides were incubated within a tightly closed humidified box overnight at 4° C. Then the glass slides were washed three times with PBST (0.05% Tween 20). Then the glass slides were blocked by adding 200 μl blocking buffer per slide and incubated at room temperature for 2 hours. Then the glass slides were similarly washed three times and added with 200 μl antigens diluted in dilution buffer and incubated at room temperature for 2 hours. Then the glass slides were similarly washed three times and added 200 μl of human Clq (2 μg/ml) per slide and incubated overnight at 4° C. Then the glass slides were similarly washed three times and added sheep-anti-human Clq polyclonal antibodies (1:200 dilution) and incubated at room temperature for 1 hour. Then the glass slides were similarly washed three times and added rabbit anti-sheep IgG polyclonal antibodies (1:2000 dilution) and incubated at room temperature for 1 hour. Then the glass slides were similarly washed three times and added substrate buffer. The coloring was stopped after 3-5 minutes, and the glass slides were scanned and photographed with Gel Imaging System (VL, France), or photographed with Canon EOS-10D camera (Canon, USA), then the pictures were converted into JPG form using Photoshop 7.0 (Adobe, USA).

First, optimised the fixing buffer. Carbonate buffer solution (CBS) (0.05 mol/L, pH9.6, with 30% of glycerol) and PBS (pH 7.4, with 30% of glycerol) were used to spot the mAb. IE6 was diluted in both fixing buffers into 40 μg/ml. The diluted antibody was spotted into a 4×4 array manually (1 μl/spot). The remaining operation was the same as the basic protocol described above. As a result, CBS provided better results. Thus, CBS would be used for all remaining experiments.

Second, optimised the blocking buffer. 3% BSA and 5% fat-free milk were tested for their blocking effects. IE6 was diluted in CBS into 40 μl/ml and spotted into a 3×3 array. Other than different blocking buffers were used, the remaining operation was the same as the basic protocol described above. Actually, most of the blocking buffers tested gave rise to similar results.

Third, optimised the fixing time of the spotted mAb. The slides were similarly prepared and processed as the Second (3% BSA) except that the spotted slides were incubated for 12 hours, 24 hours, 48 hours, and 72 hours separately. As a result, the incubation time between 24 to 48 hours provided fair results.

Fourth, optimised the effect of different concentrations of glycerol. The fixing buffer was added with 10%, 20%, 30%, and 40% of glycerol separately. The slides were similarly prepared and processed as the Second (3% BSA) and Third (24 hours for fixing). As a result, 30% glycerol in the fixing buffer provided the best results.

Finally, optimised the concentrations of spotted mAbs. IE6, IIB6C3, and IlIC2D2-01 were diluted into 400 μg/ml, 40 μg/ml, 4 μg/ml, and 0.4 μg/ml sequentially and spotted onto the glass slides with three spots for each mAb. The conditions were the optimised ones as described above. As for the array, the left-most column was the quality control (rabbit anti-sheep IgG polyclonal antibodies conjugated to HRP); the first row from the top was IE6; the second row was IIB6C3; and the bottom row was IIIC2D2-01. As a result, the concentration of 40 μg/ml was the best workable concentration.

Buffers and reagents. Coating buffer was carbonate buffer solution (CBS) (0.05 mol/L, pH9.6, with 30% glycerol). Washing buffer was 0.15 mol/L PBS (pH 7.4 with 0.05% Tween-20). Blocking buffer was 5% no-fat milk powder in PBS, 5% BSA in PBS, or 5% milk powder in PBS. Dilution buffer was washing buffer with 0.1% BSA. Substrate buffer was phosphate buffer (pH 5.0). Developing buffer was made of dissolution of TMB in substrate buffer and H₂O₂. Stopping buffer was 2 mol/L H2SO4. TMB and BSA were from Sigma (USA). 96-wellplates were from Corning (USA).

ELISA. Typical sandwich ELISA was used to detect reactivities of mAbs to relevant antigens. Briefly, mAbs were diluted in coating buffer and distributed in each well (100 μl) of a 96-well plate. The plate was incubated overnight at 4° C. and washed 3× using PBST, blocked with blocking buffer at 4° C. for 5 hours, and washed again. Antigens were diluted in dilution buffer, and added into each well. The plate was incubated at room temperature for 2 hours, and similarly washed. Then the plate was added with specific polyclonal antibody (chicken sera infected with H5N1 AIV for mAbs against H5N1 viruses, chicken sera infected with H9N2 AIV for mabs against H9N2 virus, cavy sera infected with FMDV for mAbs IIID6D6-02 and 2D5H12) and incubated 1 hour at room temperature. Then, the plate was washed, added second antibody conjugated to HRP (room temperature, 1 hour), washed, added rabbit anti-sheep IgG polyclonal antibodies (room temperature, 1 hour), washed, added TMB substrate (reaction for 5-10 minutes), stopped, and read at 450 nm.

Results

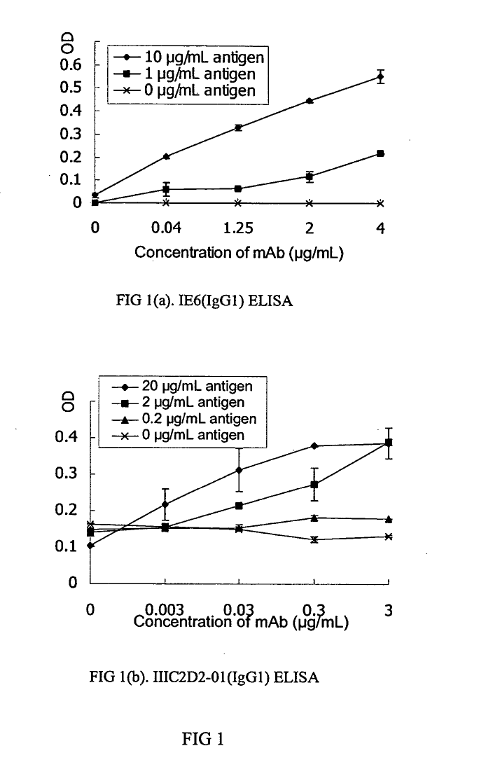

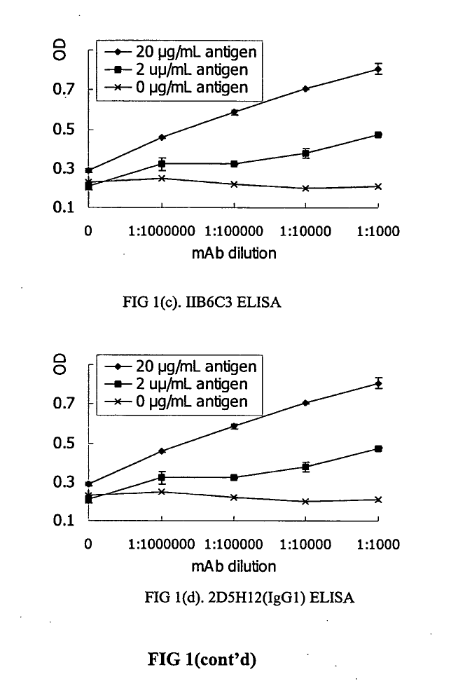

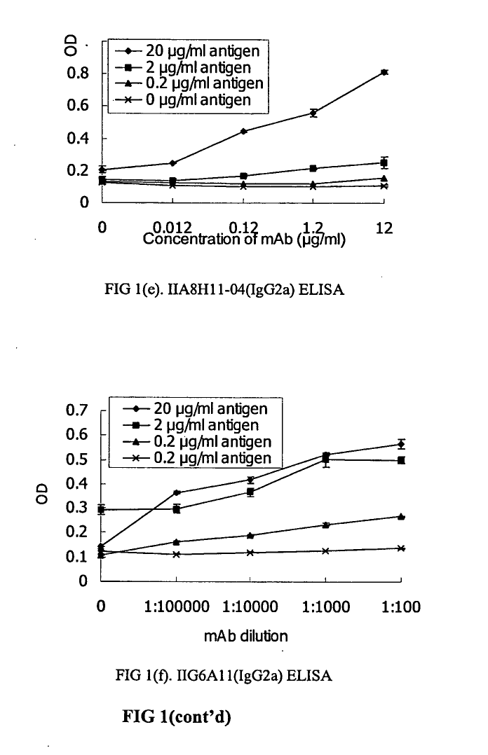

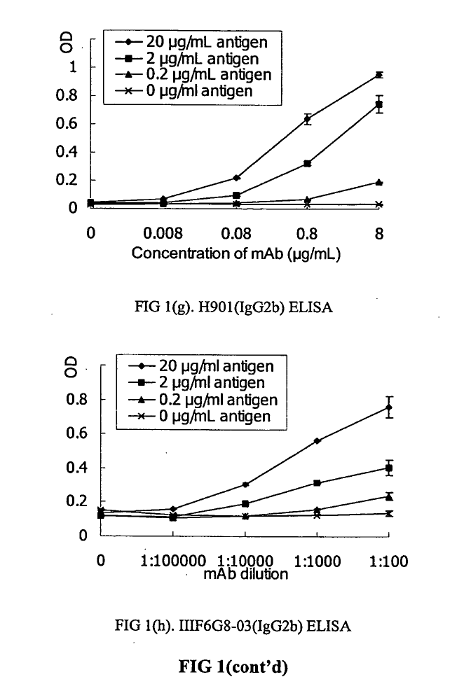

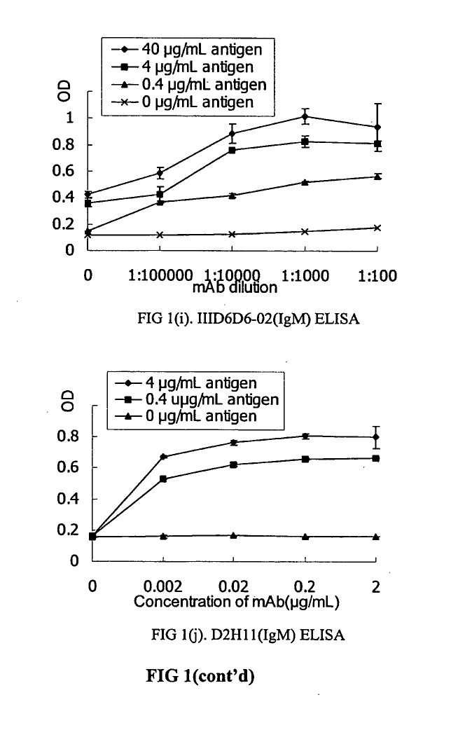

FIG. 1 summarizes the sandwich ELISA detection of antigens bound to different subclasses of murine IgG monoclonal antibodies and IgM monoclonal antibodies. (a)-(d) show the results with murine IgG1 mAbs; (e) & (f) with IgG2a mAbs; (g) & (h) with IgG2b mAbs; and (i) & (j) with IgM mAbs. The y axis represents the OD reading at 450 nm; the x axis represents the concentrations of coated antibodies (μg/ml for purified mAbs and dilution times for ascites). The concentrations of antigens used in each assay were denoted accompanying each curve. The data are given as means of triplicates with bars indicating standard deviation using Two-way ANOVA analysis. All mabs except for IIA8H11-04 showed statistical differences between all tested antigen concentrations and the empty one under all conditions (a=0.05, 0.01, 0.005, and 0.001). As for IIA8H11-04, the difference between 0.2 μg/ml and the empty one is not statistically different under all conditions.

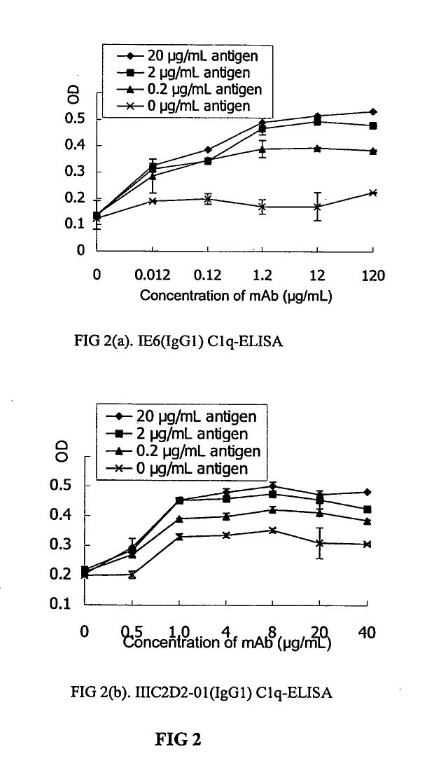

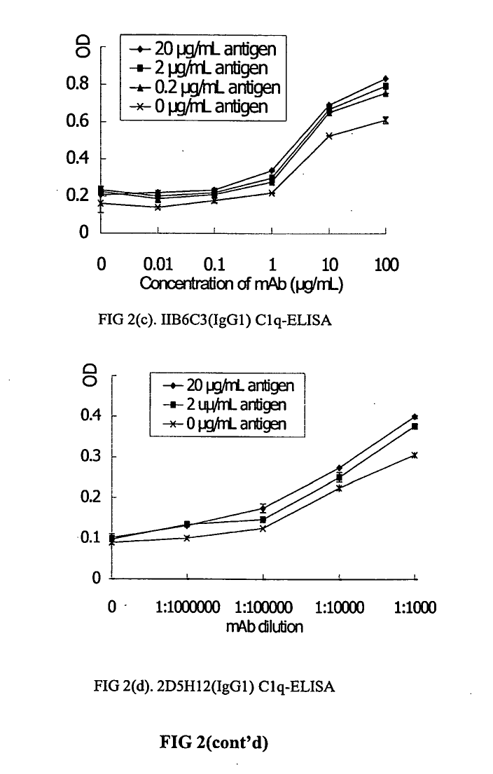

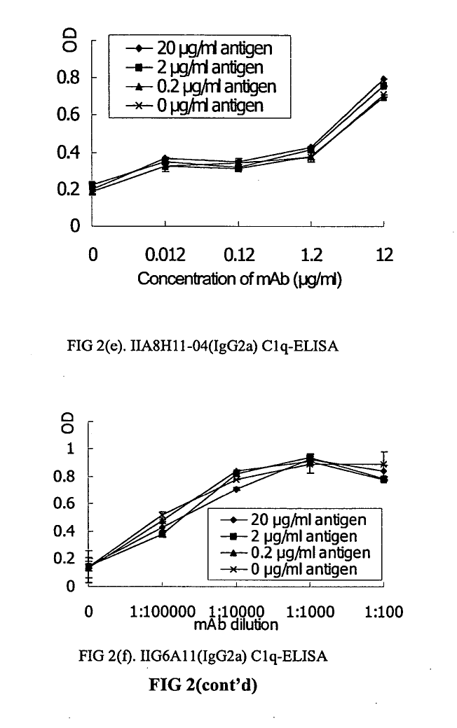

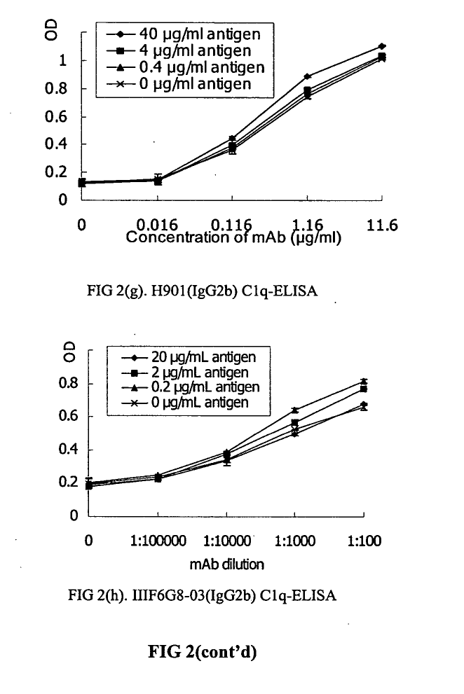

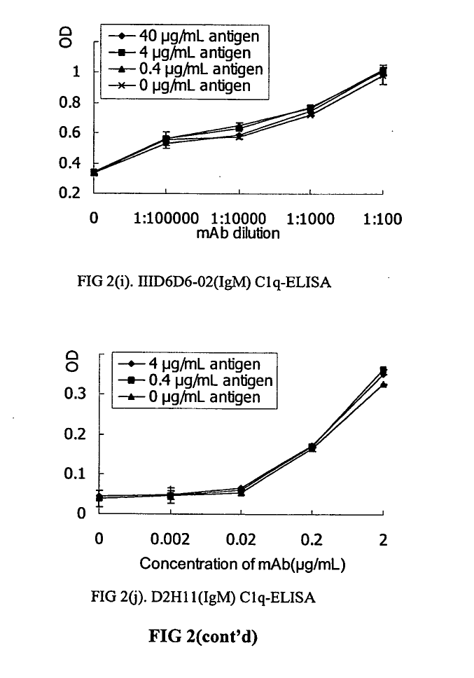

FIG. 2 summarizes the Clq-ELISA detection of antigens bound to three different subclasses of murine IgG mAbs and IgM mAbs. (a)-(d) show the results with murine IgG1 mAbs; (e) & (f) with IgG2a mAbs; (g) & (h) with IgG2b mAbs; and (i) & (j) with IgM mAbs. The y axis represents the OD reading at 450 nm; the x axis represents the concentrations of coated antibodies (μg/ml for purified mAbs and dilution times for ascites). The concentrations of antigens used in each assay were denoted accompanying each curve. The data are given as means of triplicates with bars indicating standard deviation using Two-way ANOVA analysis.

These results have shown the following apparent features. First, the binding of human Clq to IgG1, IgG2a, IgG2b, and IgM increased in parallel with the increase of the coated antibody concentrations (see all the OD readings without antigens in FIG. 2(a)-1(j)); this is in line with the literature that Clq can bind to coated antibodies with high concentrations. Second, the additions of antigens to IgM mAbs failed to increase the human Clq detection signals (see FIGS. 2(i)-1(j)). In other words, human Clq could not differentiate empty IgM mAbs from the loaded ones. There is no statistical difference between the loaded ones and the empty ones under all conditions (a=0.05, 0.01, 0.005, and 0.001). Third, human Clq could differentiate the empty IgG mAbs from the loaded ones but different IgG subclasses showed evident differences. All four IgG1 mAbs came out the best: showing statistical differences between all loaded ones and the empty ones under all conditions (a=0.05, 0.01, 0.005, and 0.001) (see FIG. 2(a)-(d)). With respect to IgG2a, IIA8H11-04 showed statistical difference over the empty one when the antigen concentrations were 2-20 μg/ml under conditions (a=0.05, 0.01, and 0.005), but only 20 μg/ml showed statistical difference under condition (a=0.001) (see FIG. 2(e)); and IIG6A11 showed no statistical differences under all conditions (a=0.05, 0.01, 0.005, and 0.001) (see FIG. 2(f)). With respect to IgG2b mAbs, H901 showed statistical difference over the empty one only when the antigen concentration was 40 μg/ml under all conditions (a=0.05, 0.01, 0.005, and 0.001) (see FIG. 2(g)); and IIIF6G8-03 showed statistical difference over the empty one under all tested antigen concentrations and all conditions (a=0.05, 0.01, 0.005, and 0.001) (see FIG. 2(h)). Finally, while we have ruled out the possibility of Clq binding to the antigens directly by directly binding of Clq to immobilized antigens (data not shown), these results farther demonstrated that human Clq did not bind directly to the antigens (H5N1 AIV, H9N2 AIV, and FMDV).

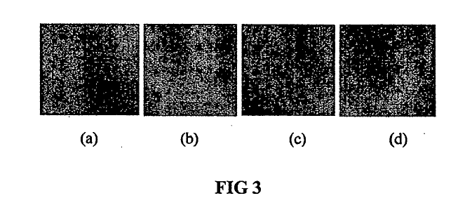

FIG. 3 summarizes the Clq-based detection of antigens bound to mAb arrays. All slides had the same array: the leftmost column is the positive control (rabbit anti-sheep IgG polyclonal antibodies conjugated to HRP); for the remaining three columns, the first row contains triplicate of IE6, the second row of IIIC2D2-01, and the third row of 2D5H12. All antibodies were spotted at 40 μg/ml. For detection assays, each slide was included with different antigens: (a) no antigen; (b) H5N1 (1 ug/ml); (c) FMDV (1 ug/ml); (d) mixture of H5N1 and FMDV (1 ug/ml).

While the present invention has been described with reference to particular embodiments, it will be understood that the embodiments are illustrative and that the invention scope is not so limited. Alternative embodiments of the present invention will become apparent to those having ordinary skill in the art to which the present invention pertains. Such alternate embodiments are considered to be encompassed within the spirit and scope of the present invention. Accordingly, the scope of the present invention is described by the appended claims and is supported by the foregoing description.

What is claimed is:

1. A method for detecting antigens captured on an antibody, comprising the following steps of:
providing at least one IgG monoclonal antibody,
contacting the at least one IgG monoclonal antibody with a sample containing at least one antigen, wherein the at least IgG monoclonal antibody is specific for the at least one antigen; and
detecting the at least one antigen captured by the at least one IgG monoclonal antibody with a detecting agent that differentially binds to the at least one IgG monoclonal antibody when it binds to the at least one antigen over the at least one IgG monoclonal antibody when it is empty; thereby when the at least one antigen binds to the at least one IgG monoclonal antibody, the detecting agent binds to the at least one IgG monoclonal antibody differentially; thus the at least one antigen can be detected by the detecting agent without discrimination between antigens.

2. The method of claim 1, wherein the at least one IgG monoclonal antibody is from species selected from the group consisting of mouse, rat, rabbit, sheep, goat, horse, bovine, dog and human.

3. The method of claim 1, wherein the at least antigen is one selected from the group consisting of proteins, peptides, glycoproteins, lipoproteins, lipids, nucleic acids, small molecules, allergens, viruses, bacteria, yeasts, fungi, unicellular/multicellular organisms, and cells.

4. The method of claim 1, wherein the sample includes a clinical sample, and cultured cells, cell supernatants, cell lysates, serum, plasma, biological fluid, and a pure or enriched bacterial or viral sample derived from any of these.

5. The method of claim 1, wherein the detecting agent is a protein or antibody or, portions or fragments thereof.

6. The method of claim 1, wherein the detecting agent is Clq or, portions or fragments thereof.

7. The method of claim 1, wherein the at least one IgG monoclonal antibody forms an array with two or more different IgG monoclonal antibodies.

8. A system for detecting antigens captured on at least one IgG monoclonal antibody, comprising:
at least one IgG monoclonal antibody, wherein the at least one IgG monoclonal antibody is specific for at least one antigen; and
a detecting agent that differentially binds to the at least one IgG monoclonal antibody when it binds to the at least one antigen over the at least one IgG monoclonal antibody when it is empty; thereby when the at least one antigen binds to the at least one IgG monoclonal antibody, the detecting agent binds to the at least one IgG monoclonal antibody differentially; thus the at least one antigen can be detected by the detecting agent without discrimination between antigens.

9. The system of claim 8, wherein the at least one IgG monoclonal antibody is from species selected from the group consisting of mouse, rat, rabbit, sheep, goat, horse, bovine, dog and human.

10. The system of claim 8, wherein the at least one antigen is one selected from the group consisting of proteins, peptides, glycoproteins, lipoproteins, lipids, nucleic acids, small molecules, allergens, viruses, bacteria, yeasts, fungi, unicellular/multicellular organisms, and cells.

11. The system of claim 8, wherein the detecting agent is a protein or antibody or, portions or fragments thereof.

12. The system of claim 8, wherein the detecting agent is Clq or, portions or fragments thereof.

13. The system of claim 8, wherein the at least one IgG monoclonal antibody forms an array with two or more different IgG monoclonal antibodies.

14. A diagnostic kit for detecting markers derived from a disease or clinical samples, comprising:
at least one IgG monoclonal antibody, wherein the at least one IgG monoclonal antibody is specific for at least one disease marker; and
a detecting agent that differentially binds to the at least one IgG monoclonal antibody when it binds to the at least one disease marker over the at least one IgG monoclonal antibody when it is empty, thereby when the at least one disease marker binds to the at least one IgG monoclonal antibody, the detecting agent binds to the at least one IgG monoclonal antibody differentially; thus the at least one disease marker can be detected by the detecting agent without discrimination between disease markers.

15. The diagnostic kit of claim 14, further comprising an instruction sheet.

16. The diagnostic kit of claim 14, wherein the detecting agent is Clq or antibody, portions or fragments thereof.

17. The diagnostic kit of claim 14, wherein the at least one IgG monoclonal antibody forms an array with two or more different IgG monoclonal antibodies.
